(19) United States
(12) Patent Application Publication
(10) Pub. No.: US 2012/0290082 A1
(43) Pub. Date: Nov. 15, 2012

(54) MECHANICAL TRANSCATHETER HEART VALVE PROSTHESIS

(75) Inventors: Bodo Quint, Oberglatt (CH); Matthias Wesselmann, Ruedlingen (CH); Alwin Schwitzer, Buelach (CH); Patrice Bachmann, Winterthur (CH); Susanne Pfenninger-Ganz, Aathal (CH); Hans Lang, Buchs (CH); Amir Fargahi, Buelach (CH)

(73) Assignee: BIOTRONIK AG, Buelach (CH)

(21) Appl. No.: 13/458,015

(22) Filed: Apr. 27, 2012

Related U.S. Application Data
(60) Provisional application No. 61/484,241, filed on May 10, 2011.

(51) Int. Cl. A61F 2/24

(52) U.S. Cl. 623/2.19

(57) ABSTRACT

A mechanical transcatheter heart valve prosthesis including a support structure that can be expanded from a first size designed for minimally invasive insertion to a functional second size and a flexible heart valve made of a composite material containing woven fabric that is embedded in an elastic matrix and locally reinforced by fibers, the heart valve comprising a plurality of leaflets.

### CROSS REFERENCE TO RELATED APPLICATIONS

This patent application claims the benefit of U.S. Provisional Patent Application No. 61/484,241, filed on May 10, 2011, which is hereby incorporated by reference in its entirety.

### TECHNICAL FIELD

The invention relates to a mechanical transcatheter heart valve prosthesis.

### BACKGROUND

The interventricular septum separates the human heart into two halves, more specifically into a right ventricle and a right atrium and a left ventricle and a left atrium. Four heart valves are located between the ventricles and atriums. The oxygen-depleted but carbon dioxide-rich blood initially flows through the tricuspid valve into the right atrium and from there into the right ventricle. The tricuspid valve is a valve having three leaflets and is also referred to as an atrioventricular valve. From the right ventricle, the blood flows through the pulmonary valves into the two lung lobes, where the blood is enriched again with oxygen. The pulmonary valve is what is called a semilunar valve. The oxygen-enriched blood then travels from the lungs into the left atrium and is pumped through the mitral valve, which has the shape of a bicuspid atrioventricular valve, into the left atrium. The blood finally travels from the left ventricle through the aortic valve into the major blood circulation system. Like the pulmonary valve, the aortic valve is a semilunar valve.

When a patient has a valvular heart defect, it can be assumed that over time the function of these heart valves can steadily worsen. After coronary bypass surgery, the replacement of heart valves that are no longer functioning with heart valve prostheses has meanwhile become the most frequent surgery on the human heart. An ideal heart valve replacement should have unlimited durability, enable unimpaired blood flow conditions in the vessel, not cause any heart valve-related complications such as thrombogenicity or susceptibility to endocarditis, not exhibit any prosthesis-immanent risks like valve-related defects, enable easy implantation, and be low-noise.

Two different types of heart valve prostheses are used, these being mechanical and biological heart valve prostheses.

Biological heart valves are prostheses that are composed of biological material, in particular the aortic valve leaflets of pigs or the pericardial sac of cows. The tissue is generally chemically treated for fixation and attached to an expandable support frame for subsequent fixation in the heart.

In the implanted state, however, biological heart valve prostheses have only limited durability because calcification increasingly influences the valves. An average service life of approximately 10 to 12 years is to be expected. Fast calcification of the biological heart valves is particularly pronounced in younger patients. The implantation of biological heart valves is therefore only performed in older patients to prevent a second replacement of the valve. In addition, the chemical treatment of natural tissue for biological heart valves requires considerable efforts to preserve the mechanical properties. The BSE problem additionally demonstrates that the use of biomaterials also has inherent risks.

Compared to the biological heart valves, mechanical heart valves have the advantage of greater durability. A mechanical heart valve replacement, however, necessitates lifelong anti-coagulation treatment.

Meanwhile, artificial heart valves have been developed that have a proven service life in the laboratory of 150 years. The hemodynamic conditions are approximately comparable to those of natural heart valves. Valve-related complications such as the formation of clots have virtually disappeared, and prosthesis-related complications have been practically eliminated due to outstanding refinements. The mechanical heart valves are relatively easy to implant, and the valve noise of the mechanical heart valve is being tolerated well by patients because these valves are relatively low in noise—at least for the surroundings.

A variety of the presently approved artificial heart valves, however, can only be implanted through open-heart surgery, which clearly limits the use of these prostheses, because the surgical risk is high in slightly more than ⅓ of all patients or such surgery is not even possible. For this reason, minimally invasive techniques and transcatheter heart valve prostheses have meanwhile been developed, wherein the new heart valve is placed and anchored at the implantation site using a catheter system (for example, PAVR, percutaneous aortic valve replacement). The anchoring in the vessel wall is carried out using a support structure for the actual heart valve, for example a metallic mesh, the design and material selection of which are based on that of a stent. The support structure can be self-expanding or is expanded using a balloon catheter.

Conventional transcatheter heart valve prostheses therefore have a support structure, which can be expanded from a first size, which is designed for minimally invasive insertion, to a functional second size. The actual heart valve, which initially takes on a first shape that is designed for minimally invasive insertion and can be expanded over the course of the implantation to the functional second shape, is fixed to this support structure. For example, the heart valve comprises a plurality of flexible leaflets, which open or close depending on the acting blood flow forces. Such a transcatheter heart valve prostheses comprising a biological heart valve is disclosed in EP 1 267 753 B1, for example.

In practical experience, it is necessary to provide transcatheter heart valve prostheses in a wide variety of sizes, because the required valve diameters differ considerably among the recipients. The dimension of the valve structure for a flexible heart valve must therefore be adapted very precisely to the assembly diameter, so as to prevent the formation of folds as well as leaks. To this end, care must be taken that any deformation work that must be performed to close the valve can lead to a loss of pump volume. Until now, this disadvantage had to be tolerated when adapting known heart valves to the dimensions required at the implantation site.

### SUMMARY

It is the object of the present invention to mitigate or prevent one or more disadvantages of the prior art. It is in particular an object of the present invention to provide a mechanical transcatheter heart valve prosthesis, which is composed of an artificial material and exhibits mechanical behavior that is optimized for the intended purpose.

The present invention achieves this object by providing a mechanical transcatheter heart valve prosthesis, which comprises a support structure that can be expanded from a first size designed for minimally invasive insertion to a functional second size and a flexible heart valve made of a composite material containing woven fabric that is embedded in an elastic matrix and locally reinforced by fibers, the heart valve comprising a plurality of leaflets.

The invention is based on the concept of designing a heart valve so that viscoelastic effects can be utilized to achieve automatic adaptation to the vessel dimensions. The effect is based on stress relief in the regions that are decisively characterized by closing forces of the heart valve or absorb these. The basic structure of the heart valve, however, must be preserved in the pressure-loaded state—this object is achieved in the present case by local fiber reinforcement in special (these) regions. To this end, according to the invention the flexible heart valve fastened to a conventional support structure is made of an artificial material, which is woven fabric-based. This woven fabric is embedded in a viscoelastic material so as to produce a closed, sealed membrane. The local fiber reinforcement of the heart valve in regions in which high stresses are to be expected during functional use allows the heart valve to be stiffened or viscoelastic deformation to be suppressed. In regions that are not effectively fiber-reinforced, the viscoelastic matrix material can be shaped by viscoelastic stretching, a retardation phenomenon, in the pressure-loaded state. Using textile principles, it is possible to produce substrates that exhibit advantageous anisotropic properties. For example, woven fabrics having fiber arrangements that can be stretched in one direction and are consequently “adaptable”, but cannot be stretched in another direction and consequently remain pressure-resistant and dimensionally stable, can be used as the substrate for production.

With polymers as well as metallic constructs, it is assumed that the hysteresis between the loaded state and relieved state denotes energy absorption by the material, which limits the time-based strain capacity of the system. On the other hand, the viscoelasticity of polymeric materials also offers the possibility to adapt a technical construct to dimensions. In the case of the heart valve, which in the closed state is under high mechanical stress, and in this state can thus be characterized by viscoelasticity, this technical material property can be deliberately used to achieve dimensional adaptation. An isotropic viscoelastic valve material, however, would yield to the load stress of the heart valve in the long term. For this reason, an anisotropic construct may be required, which has the freedom to adapt, yet the anisotropy of which should be such that it does not enable deformations, or enables only minimal deformations, in deformation directions that are essential for the blocking function of the heart valve. In other words, ideally regions of the artificial heart valve that are deformed during the dimensioning should exhibit maximum adaptability. In contrast, the regions of the heart valve that experience particular stress after the dimensional adaptation to the intended function has been carried out should maintain the dimensions thereof, so as to ensure long-term function of the artificial structure. Moreover, there may be the need to locally influence both the stiffness and the strength of the artificial heart valves. To ensure the minimally invasive application, materials having maximum strength should always be combined with materials having very high strength and stretching, whereby the mechanical function can be achieved with a minimum layer thickness.

According to a preferred embodiment of the invention, the heart valve is reinforced by the fibers in an edge section adjoining the support structure. Furthermore, it is preferred for the heart valve to have a tricuspid contour.

Other aspects that are essential with reference to the teaching according to the invention:
a) The typical property of a nitinol design-based support structure, which exerts significant forces on the vascular wall, is a migration of the structure into deeper regions of the tissue. This can cause the dimension and positioning of the support structure in the vessel to become critical because deviations in the diameter directly affect the ability of the valve structure to close. The heart valves are located in the level of connective tissue referred to as the cardiac skeleton, which is able to limit the migration well. When this level is left, the tolerance with respect to migration is considerably lower.
b) Reproducing the heart valve structure from a homogeneous material necessitates the use of an elastic polymer. Elastomers, however, always exhibit viscoelastic behavior, which limits the deformation resistance thereof. Elastomers having the most ideal elastic behavior possible (which is to say, low viscoelasticity) are generally implemented in fiber composites or steel composites in many technical applications, so as to ensure the deformation resistance thereof. While it may be desirable for the individual flexible leaflets of a heart valve to be oriented and adapt to each other, the deformation resistance of the membrane segment, which decisively absorbs the forces in the closed state of the heart valve, is also required to ensure the functional shape over an extended period of time.
c) To enable the valve to close and open at very lower pressure differential, the valve must be able to undergo very high bending locally. It may be necessary to deliberately provide these regions with a reinforcement, which should exhibit both high strength and ideal elastic behavior. Because of the requirement to implement this spatially with very low material thicknesses, the path of a fiber reinforcement is advantageous from a technical perspective.
d) In order to define movement points and bending points for an advantageous valve behavior, regions of the heart valve can be deliberately thickened and stiffened by way of textiles using fibers.

The woven fabric used in the composite material according to the invention in the present case is understood to be a textile sheet material composed of at least two different fiber orientations. This can be achieved by a woven fabric structure or individual fiber layers, because it is assumed that the final composite is ensured by a polymeric matrix. The term ‘woven fabric’ covers in particular crossed fiber systems, but also a product that is yielded by intertwining a plurality of strands of flexible material (meshwork).

The woven fabric is fixed in the product by way of a polymeric matrix. The matrix is preferably composed of a polyurethane, because polyurethanes generally constitute an excellent compromise of strength and elasticity. These properties are notably necessary to achieve small dimensions, for example, thin layer thicknesses.

The fiber reinforcement according to the invention can be done using known technologies. For example, textile fiber placement technologies and nitriding processes are known. In order to obtain a stable textile base body that is as thin as possible, high-strength fibers and fibers that are resistant to continuous stress can be used. The fibers preferably comprise high-strength polyethylene (PE). It is possible, for example, to use PE fibers sold by the name of Dyneema, including for implant applications (available from DSM N.V.).

The composite material may further comprise a coating to improve the biocompatibility. The coating comprises or consists of a biocompatible material, which improves cell seeding, for example. A biocompatible material is a non-living material, which is employed for applications in medicine and interacts with biological systems. A basic prerequisite for the use of a material that is in contact with the surrounding body area when used as intended is the biocompatibility thereof (body friendliness). Biocompatibility shall be understood as the ability of a material to evoke an appropriate tissue response in a specific application. This includes an adaptation of the chemical, physical, biological, and morphological surface properties of an implant to the recipient's tissue with the aim of a clinically desirable interaction. Biocompatible materials that are preferred for the coating are substantially bioinert. Bioinert materials shall be understood as those materials which after implantation remain substantially intact over the intended service life of the heart valve prosthesis and exhibit no significant biocorrosion. A possible test medium for testing the corrosion behavior of a potential material is synthetic plasma, as that which is required according to EN ISO 10993-15:2000 for biocorrosion analyses (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the material to be analyzed is stored in a closed sample container with a defined quantity of the test medium at 37° C. The samples are removed at intervals—which are matched to the anticipated corrosion behavior—ranging from a few days to several months or years and analyzed for traces of corrosion in the known manner. The synthetic plasma according to EN ISO 10993-15:2000 corresponds to a blood-like medium and thus is a possible medium to reproducibly simulate a physiological environment as defined by the invention. A material is considered to be bioinert notably when the material has corroded less than 10% in the above-described test after a period of 12 months.

### DESCRIPTION OF THE DRAWINGS

The invention will be explained in more detail hereinafter based on one exemplary embodiment and the related figures. In the figures:

FIG. 1 shows the composite material in one embodiment.

FIG. 2 illustrates the cut of the composite material of FIG. 1.

FIG. 3 sketches the three-dimensional deformation of the material for producing the heart valve.

FIG. 4 visualizes the geometric limitations on the flat structure.

### DETAILED DESCRIPTION

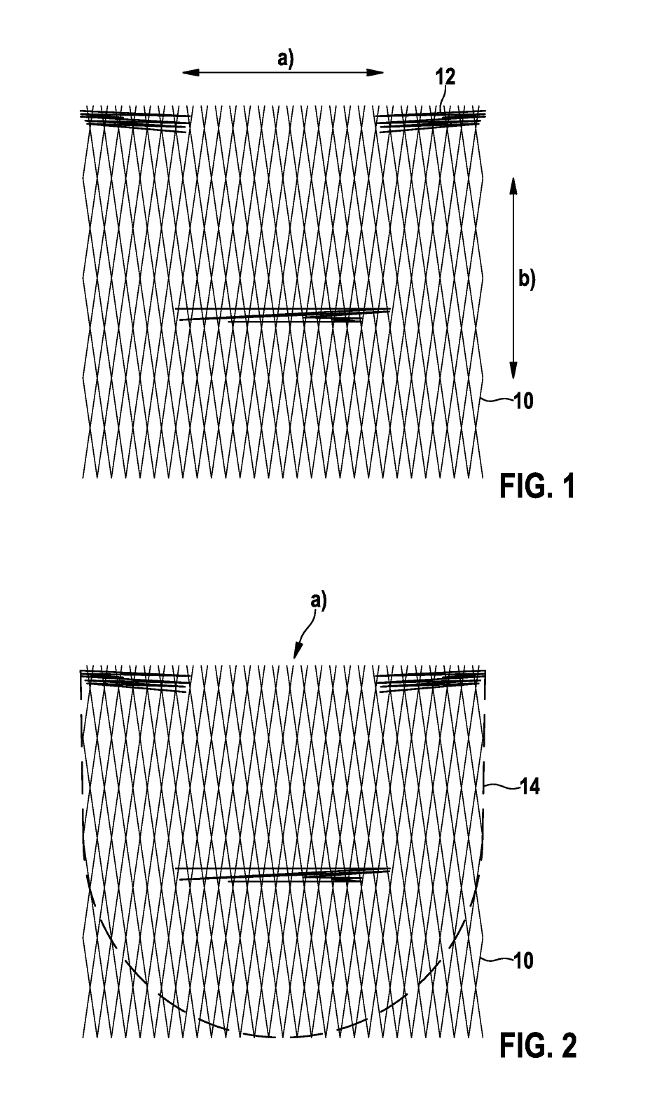

FIG. 1 illustrates a composite material, which has anisotropic mechanical properties. The composite material comprises a woven fabric 10, which is fixed by an elastomeric matrix. The woven fabric 10 is more elastic in direction a) than in direction b). If the upper edge of this structure is also to form the upper edge of a flexible heart valve composed of a plurality of leaflets, a large part of the bending of the valve as well as the force transmission to the valve base would be concentrated in the lateral regions. Analogously, a laid fiber scrim can be used instead of a woven fabric.

For this reason, fibers 12 are drawn into the woven fabric 10 with the illustrated orientation. A central region remains between the upper end regions, which are fiber-reinforced, with this central region remaining stretchable in direction a)—however the stretching thereof in direction b) is highly limited by the fibers 12.

The cylindrical shape of the heart valve is to form under pressure in the central to lower region of the woven fabric 10. Assuming that the heart valve should be fixed in the edge regions and the woven fabric 10 can transfer forces from the upper region to the center, it is useful to likewise strengthen the central region in part by a fiber reinforcement.

In this way a lower region of the heart valve is obtained, wherein the fibers 12 extend completely through the structure thereof and the deformation is limited. This lower region is intended to bear the pressure load of the closed heart valve without viscoelastic deformation. In this way, a composite material can be provided, which comprises regions that can be viscoelastically deformed and are stable to stresses even over extended periods.

In order to form the heart valve, the composite material must be cut to size along the cutting line 14 of FIG. 2.

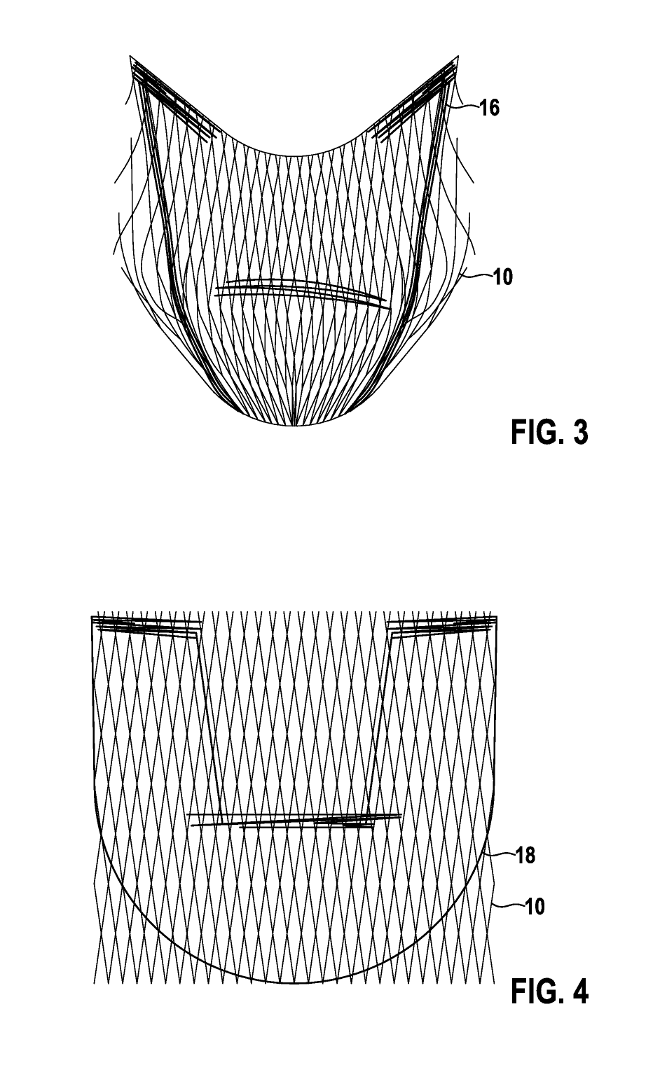

FIG. 3 illustrates a deformation of the composite material downward and a deformation toward the 120° angle of the upper heart valve edge. Line 16 represents the region in which the heart valve will later be connected to a cylindrical support structure.

In FIG. 4, a line 18 visualizes the geometric limitations on the flat structure.

The described fiber reinforcement is intended to improve the mechanical long-term stability of the heart valve. However, the local reinforcement may also cause stiffening, which can be used to improve dynamic motion mechanics. The bending regions of the individual leaflets of the heart valve are concentrated in the edge regions of the heart valve so that the diameter that is exposed when the heart valve opens is as large as possible.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A mechanical transcatheter heart valve prosthesis, comprising:
a support structure, which expands from a first size designed for minimally invasive introduction to a functional second size; and
a flexible heart valve comprising a composite material containing woven fabric that is embedded in an elastic matrix and locally reinforced by fibers, the heart valve comprising a plurality of leaflets.

2. The heart valve prosthesis according to claim 1, wherein the heart valve is reinforced by the fibers in an edge section adjoining the support structure.

3. The heart valve prosthesis according to claim 1, wherein the heart valve has a tricuspid contour.

4. The heart valve prosthesis according to claim 1, wherein the matrix comprises polyurethane.

5. The heart valve prosthesis according to claim 1, wherein the fibers comprise high-strength polyethylene (PE).
